(19) United States
(12) Patent Application Publication
(10) Pub. No.: US 2019/0327414 A1
(43) Pub. Date: Oct. 24, 2019

(54) IMAGING DEVICE, ENDOSCOPE, AND ENDOSCOPE SYSTEM

(71) Applicant: OLYMPUS CORPORATION, Tokyo (JP)

(72) Inventor: Susumu YAMAZAKI, Tokyo (JP)

(73) Assignee: OLYMPUS CORPORATION, Tokyo (JP)

(21) Appl. No.: 16/456,538

(22) Filed: Jun. 28, 2019

Related U.S. Application Data
(63) Continuation of application No. PCT/JP2017/030785, filed on Aug. 28, 2017.

(30) Foreign Application Priority Data
Jan. 24, 2017 (JP) 2017-010117

(51) Int. Cl. H04N 5/232; A61B 1/00

(52) U.S. Cl. CPC H04N 5/23241; H04N 2005/2255; A61B 1/00121; A61B 1/00009

(57) ABSTRACT

An imaging device includes: a pulse-signal superimposing circuit configured to superimpose a first pulse signal generated by a pulse-signal generator on a voltage; a separating circuit that is disposed between a light receiver and a transmission cable, the separating circuit being configured to separate an offset voltage and a pulse voltage from the voltage transmitted through the transmission cable and output the offset voltage to the light receiver; a pulse-signal detector configured to detect the first pulse signal superimposed on the transmission cable; a pulse-signal converting circuit configured to convert a frequency of the first pulse signal detected by the pulse-signal detector into a frequency of a second pulse signal used to generate a synchronization signal for driving the light receiver; and a timing generator configured to generate the synchronization signal based on the second pulse signal obtained after conversion by the pulse-signal converting circuit.

### CROSS-REFERENCE TO RELATED APPLICATION

This application is a continuation of PCT International Application No. PCT/JP2017/030785, filed on Aug. 28, 2017, which designates the United States, incorporated herein by reference, and which claims the benefit of priority from Japanese Patent Application No. 2017-010117, filed on Jan. 24, 2017, incorporated herein by reference.

### BACKGROUND

### 1. Technical Field

The present disclosure relates to an imaging device that captures an object and generates image data on the object, an endoscope, and an endoscope system.

### 2. Related Art

In the related art, an endoscope inserts a flexible insertion portion having an elongated shape and having an imaging device provided at the distal end thereof into the body of a subject, such as patient, thereby acquiring an in-vivo image inside the body of the subject. An imaging unit used in the endoscope includes: a semiconductor chip having an imaging element formed thereon; and a circuit substrate that is disposed adjacent to the back surface side of the semiconductor chip (see Patent Japanese Patent No. 4575698 and Japanese Patent No. 4441305). In the circuit substrate, a low-pass filter, a high-pass filter, and the like, formed of a resistor and a capacitor are installed to detect synchronization signals that are output from a processor and cause the imaging element to be driven.

However, according to the above-described Japanese Patent No. 4575698 and Japanese Patent No. 4441305, the high time constants of the low-pass filter and the high-pass filter and the large values of a resistor and a capacitor included in the low-pass filter and the high-pass filter cause an increase in the size of the circuit substrate and obstructs a reduction in the size of the imaging element.

The disclosure has been made in consideration of the foregoing, and it has an object to provide an imaging device, an endoscope, and an endoscope system capable of achieving a further size reduction.

### SUMMARY

In some embodiments, an imaging device includes: a light receiver that includes a plurality of pixels that is arranged in a two-dimensional matrix to receive light from outside and generate an imaging signal corresponding to an amount of received light; a transmission cable through which electric power is transmitted to the light receiver; a power source configured to supply a voltage to the light receiver via the transmission cable; a pulse-signal generator configured to generate a first pulse signal; a pulse-signal superimposing circuit configured to superimpose the first pulse signal generated by the pulse-signal generator on the voltage; a separating circuit that is disposed between the light receiver and the transmission cable, the separating circuit being configured to separate an offset voltage and a pulse voltage from the voltage transmitted through the transmission cable and output the offset voltage to the light receiver; a pulse-signal detector configured to detect the first pulse signal superimposed on the transmission cable; a pulse-signal converting circuit configured to convert a frequency of the first pulse signal detected by the pulse-signal detector into a frequency of a second pulse signal used to generate a synchronization signal for driving the light receiver; and a timing generator configured to generate the synchronization signal based on the second pulse signal obtained after conversion by the pulse-signal converting circuit.

In some embodiments, an endoscope includes: the imaging device; an insertion portion that is insertable into a body of a subject; and a connector portion that is attachable to and detachable from an image processing device configured to perform image processing on the imaging signal. The light receiver, the separating circuit, the pulse-signal detector, the pulse-signal converting circuit, and the timing generator are located at a distal end side of the insertion portion. The connector portion includes the power source and the pulse-signal superimposing circuit.

In some embodiments, an endoscope system includes: the endoscope; and an image processing device configured to perform image processing on the imaging signal.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram that schematically illustrates the overall configuration of an endoscope system according to a first embodiment of the disclosure;

FIG. 2 is a block diagram that illustrates the function of a relevant part of the endoscope system according to the first embodiment of the disclosure;

FIG. 3 is a timing chart that illustrates the timing of each signal in the endoscope according to the first embodiment of the disclosure;

FIG. 4 is a diagram that illustrates the relationship between the filter property of a pulse-signal detector and the frequency component of a first pulse signal according to the first embodiment of the disclosure;

FIG. 5 is a block diagram that illustrates the function of a relevant part of an endoscope system according to a second embodiment of the disclosure; and

FIG. 6 is a timing chart that illustrates the timing of each signal in the endoscope according to the second embodiment of the disclosure.

### DETAILED DESCRIPTION

An endoscope system including an endoscope having an imaging element disposed at the distal end of an insertion portion, which is inserted into the subject, is explained below as an aspect (hereafter, referred to as “embodiment”) for carrying out the disclosure. Furthermore, the disclosure is not limited to the embodiment. Furthermore, the same components are attached with the same reference numeral in the description of the drawings. Further, it should be noted that the drawings are schematic and the relation between members in thickness or width, the proportion between members, and the like, differ from reality. Moreover, the drawings contain parts that are different in dimension or proportion from each other.

### First embodiment

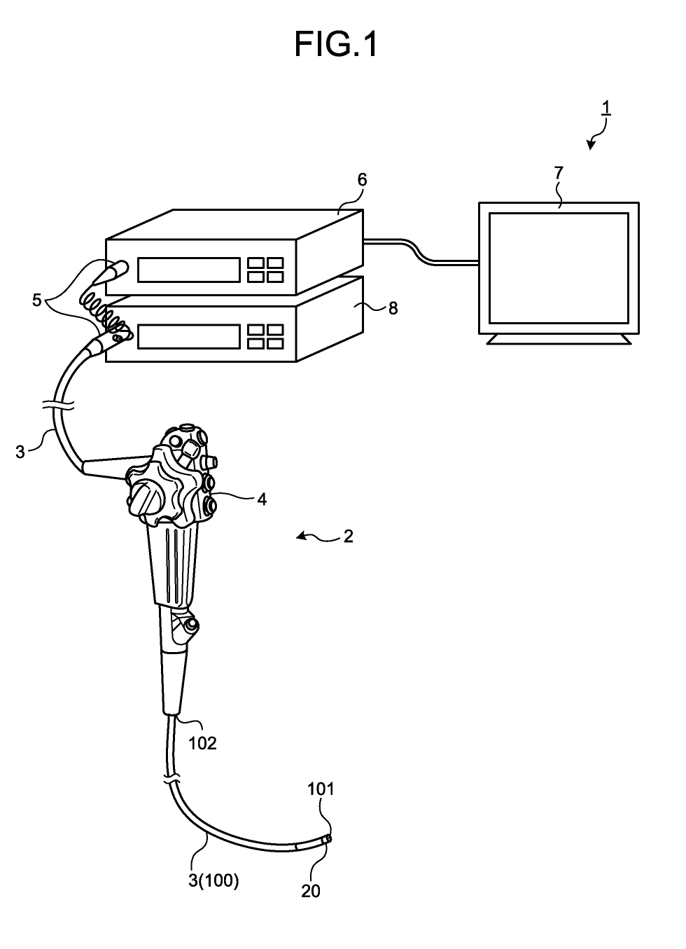

Configuration of the endoscope system FIG. 1 is a schematic diagram that schematically illustrates the overall configuration of the endoscope system according to a first embodiment of the disclosure. An endoscope system 1 illustrated in FIG. 1 includes an endoscope 2, a transmission cable 3, a connector portion 5, a processor 6, a display device 7, and a light source device 8.

The endoscope 2 inserts an insertion portion 100, which is part of the transmission cable 3, into the body cavity of the subject to capture the inside of the body of the subject and output an imaging signal to the processor 6. Furthermore, in the endoscope 2, an imaging unit 20 (imaging element) that captures in-vivo images is provided at one end side of the transmission cable 3 and at the side of a distal end 101 of the insertion portion 100 inserted into the body cavity of the subject, and an operating unit 4 that receives various operations for the endoscope 2 is provided at the side of a proximal end 102 of the insertion portion 100. Imaging signals on an image captured by the imaging unit 20 are output to the connector portion 5 via the transmission cable 3 having a length of, for example, several meters.

The transmission cable 3 couples the endoscope 2 to the connector portion 5 and couples the endoscope 2 to the light source device 8. Furthermore, the transmission cable 3 transmits imaging signals generated by the imaging unit 20 to the connector portion 5. The transmission cable 3 is configured by using a cable, optical fibers, or the like.

The connector portion 5 is coupled to the endoscope 2, the processor 6, and the light source device 8, and it executes predetermined signal processing on imaging signals output from the coupled endoscope 2, converts analog imaging signals into digital imaging signals (A/D conversion), and outputs them to the processor 6.

The processor 6 executes predetermined image processing on imaging signals input from the connector portion 5 and outputs them to the display device 7. Furthermore, the processor 6 controls the overall endoscope system 1 in an integrated manner. For example, the processor 6 performs control to switch the illumination light output from the light source device 8 and switch the imaging mode of the endoscope 2. Moreover, according to the first embodiment, the processor 6 functions as an image processing device.

The display device 7 displays an image corresponding to the imaging signal on which the processor 6 has executed image processing. Furthermore, the display device 7 presents various types of information related to the endoscope system 1. The display device 7 is configured by using a display panel of liquid crystal, organic EL (Electro Luminescence), or the like.

The light source device 8 emits illumination light from the side of the distal end 101 of the insertion portion 100 in the endoscope 2 toward the object via the connector portion 5 and the transmission cable 3. The light source device 8 is configured by using a white LED (Light Emitting Diode), which emits white light, and an LED, or the like, which emits special light that is narrow-band light having a wavelength band narrower than the wavelength band of white light. The light source device 8 emits white light or narrow-band light toward the object via the endoscope 2 under the control of the processor 6.

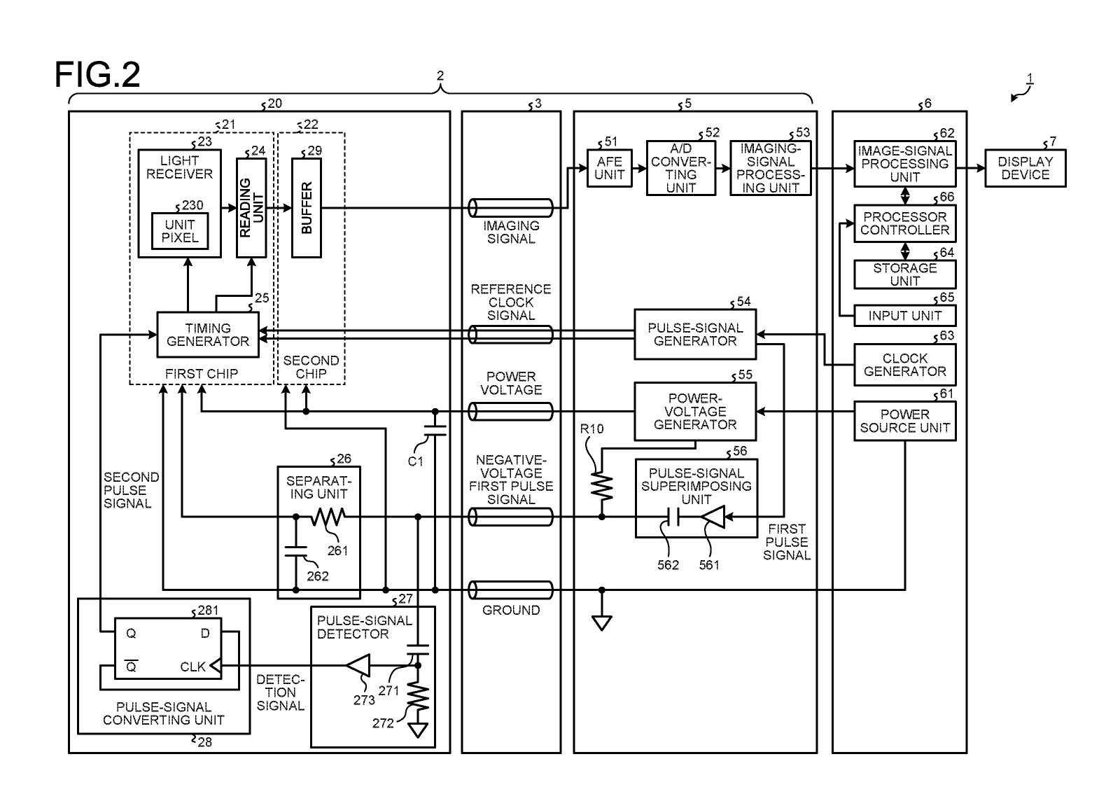

FIG. 2 is a block diagram that illustrates the function of a relevant part of the endoscope system 1. With reference to FIG. 2, an explanation is given of the details on each component of the endoscope system 1 and the path of an electric signal in the endoscope system 1.

Configuration of the Endoscope

First, the configuration of the endoscope 2 is explained. The endoscope 2 illustrated in FIG. 2 includes the imaging unit 20, the transmission cable 3, and the connector portion 5.

The imaging unit 20 includes a first chip 21 (imaging substrate), a second chip 22 (circuit substrate), a separating unit 26 (AC-component removing unit), a pulse-signal detector 27, and a pulse-signal converting unit 28. A capacitor Cl for power stabilization is disposed between a power voltage VDD to be supplied to the imaging unit 20 and a ground GND.

The first chip 21 includes: a light receiver 23 having a plurality of unit pixels 230 that is arranged in a two-dimensional matrix to receive light from outside and generate and output an imaging signal corresponding to the amount of received light; a reading unit 24 that reads an imaging signal obtained after photoelectric conversion by each of the unit pixels 230 in the light receiver 23; and a timing generator 25 that generates a synchronization signal including a light-receiver drive signal for driving the light receiver 23 and a reading-unit drive signal for driving the reading unit 24 based on a reference clock signal input from the connector portion 5 and a second pulse signal input via the pulse-signal detector 27 and the pulse-signal converting unit 28 described later and that outputs it to the light receiver 23 and the reading unit 24.

The second chip 22 includes a buffer 29 that amplifies an imaging signal output from each of the unit pixels 230 in the first chip 21 and outputs it to the transmission cable 3.

The separating unit 26 is connected between the first chip 21 and the transmission cable 3 to separate an offset voltage and a pulse voltage from the negative voltage transmitted from the transmission cable 3 and output the separated offset voltage to the first chip 21. The separating unit 26 includes: a resistor 261 (e.g., 100Ω), described later, serially connected to the transmission cable 3 (signal line) through which the negative voltage is transmitted; and a capacitor 262, described later, connected between a power-voltage generator 55 and the ground GND, and it forms an RC circuit (low-pass filter circuit). Thus, the pulse signal of a pulse voltage superimposed on the negative voltage input from the connector portion 5 described later is cut so that an offset voltage is output to the unit pixel 230.

The pulse-signal detector 27 is coupled between the separating unit 26 and a pulse-signal superimposing unit 56, described later, in the connector portion 5 due to AC coupling to detect a first pulse signal (pulse voltage) superimposed on the negative voltage and output the detected first pulse signal to the pulse-signal converting unit 28. Specifically, the pulse-signal detector 27 is coupled to the distal end side of the transmission cable 3 and the proximal end side of the resistor 261 in the separating unit 26. The pulse-signal detector 27 includes: a capacitor 271 coupled to the transmission cable 3 (signal line) through which the negative voltage is transmitted; a resistor 272 having one end side coupled to the capacitor 271 and the other end side coupled to the ground GND; and an amplifier 273 that amplifies a pulse signal extracted by the capacitor 271 and the resistor 272. The capacitor 271 and the resistor 272 form an RC circuit (high-pass filter).

The pulse-signal converting unit 28 converts the frequency of the first pulse signal detected by the pulse-signal detector 27 into the frequency of the second pulse signal used to generate a synchronization signal for driving the light receiver 23 and outputs it to the timing generator 25. The pulse-signal converting unit 28 includes a frequency divider circuit 281 that divides the frequency of the first pulse signal by an integer larger than one to convert (frequency dividing) it into the second pulse signal. That is, the frequency divider circuit 281 decreases the frequency of the first pulse signal by ½, thereby converting it into the second pulse signal.

The transmission cable 3 is configured by using at least five signal lines, i.e., the signal line for transmitting the power voltage generated by the power-voltage generator 55 to the imaging unit 20, the signal line for transmitting the negative voltage generated by the power-voltage generator 55 to the imaging unit 20, the signal line for transmitting the reference clock signal generated by a pulse-signal generator 54 to the imaging unit 20, the signal line for transmitting the imaging signal generated by the imaging unit 20 to the connector portion 5, and the signal line for transmitting the ground GND to the imaging unit 20.

The connector portion 5 includes an analog front-end unit 51 (hereinafter referred to as “AFE unit 51”), an A/D converting unit 52, an imaging-signal processing unit 53, the pulse-signal generator 54, the power-voltage generator 55, and the pulse-signal superimposing unit 56.

The AFE unit 51 receives an imaging signal propagated from the imaging unit 20, extracts a pulse voltage by using a capacitor after conducting impedance matching by using a passive element such as a resistor, and then determines the operating point by using a voltage-dividing resistor. Then, the AFE unit 51 amplifies the imaging signal (analog signal) and outputs it to the A/D converting unit 52.

The A/D converting unit 52 converts the analog imaging signal input from the AFE unit 51 into a digital imaging signal and outputs it to the imaging-signal processing unit 53. example an FPGA (Field Programmable Gate Array), and it performs a process such as noise removal or format conversion process, on digital imaging signals input from the A/D converting unit 52 and outputs it to the processor 6.

The pulse-signal generator 54 generates a reference clock signal, which is a reference for each component in the imaging unit 20, based on a clock signal (e.g., a clock signal of 27 MHz), which is supplied from the processor 6 and is a reference for an operation of each component in the endoscope 2, and outputs the reference clock signal to the timing generator 25 of the imaging unit 20 via the transmission cable 3. Furthermore, the pulse-signal generator 54 generates a first pulse signal having a pulse width shorter than that of a pulse signal for generating a drive signal (synchronization signal) for the imaging unit 20 based on the clock signal, which is supplied from the processor 6 and is a reference for an operation of each component in the endoscope 2, and outputs it to the pulse-signal superimposing unit 56. Specifically, the pulse-signal generator 54 generates the first pulse signal having a high frequency based on the clock signal, which is supplied from the processor 6 and is a reference for an operation of each component in the endoscope 2, and outputs it to the pulse-signal superimposing unit 56.

The power-voltage generator 55 is disposed on the proximal end side of the transmission cable 3 to generate the power voltage needed to drive the first chip 21 and the second chip 22 from the power supplied from the processor 6 and output it to the first chip 21 and the second chip 22. Furthermore, the power-voltage generator 55 generates the negative voltage needed to drive the unit pixel 230 in the first chip 21 from the power supplied from the processor 6 and outputs the negative voltage to the first chip 21 via the transmission cable 3. The power-voltage generator 55 generates the power voltage and the negative voltage needed to drive the first chip 21 and the second chip 22 by using a regulator, or the like. Furthermore, according to the first embodiment, the power-voltage generator 55 functions as a power source (negative power source).

The pulse-signal superimposing unit 56 is disposed on the proximal end side of the transmission cable 3 to amplify the first pulse signal (e.g., 0.5 V on the plus side) supplied from the pulse-signal generator 54, superimposes the first pulse signal onto the transmission cable 3 for transmitting the negative voltage via a resistor R10, and outputs it to the imaging unit 20. The pulse-signal superimposing unit 56 includes: an amplifier 561 that amplifies the first pulse signal supplied from the pulse-signal generator 54; and a capacitor 562 that superimposes the first pulse signal onto the negative voltage.

Configuration of the processor Next, the configuration of the processor 6 is explained.

The processor 6 is a control device that controls the entire endoscope system 1 in an integrated manner. The processor 6 includes a power source unit 61, an image-signal processing unit 62, a clock generator 63, a storage unit 64, an input unit 65, and a processor controller 66.

The power source unit 61 generates a power voltage and supplies the generated power voltage to the ground GND and the power-voltage generator 55 in the connector portion 5.

The image-signal processing unit 62 performs image processing, such as synchronization processing, white balance (WB) adjustment processing, gain adjustment processing, gamma correction processing, digital/analog (D/A) conversion processing, or format conversion processing on digital imaging signals on which the imaging-signal processing unit 53 has performed signal processing so as to convert it into an image signal and outputs the image signal to the display device 7.

The clock generator 63 generates a clock signal that is a reference for an operation of each component in the endoscope system 1 and outputs the clock signal to the pulse-signal generator 54.

The storage unit 64 stores various types of information about the endoscope system 1, data in processing, and the like. The storage unit 64 is configured by using a recording medium such as Flash memory or RAM (Random Access Memory).

The input unit 65 receives input of various operations related to the endoscope system 1. For example, the input unit 65 receives input of a command signal for switching the type of illumination light output from the light source device 8. The input unit 65 is configured by using, for example, a cross-shaped switch or a push button.

The processor controller 66 controls each unit included in the endoscope system 1 in an integrated manner. The processor controller 66 is configured by using a CPU (Central Processing Unit), or the like. The processor controller 66 switches the illumination light output from the light source device 8 in accordance with a command signal input from the input unit 65.

With the above configuration of the imaging unit 20, the negative voltage supplied from the power-voltage generator 55 is used to drive the unit pixel 230, and a small amount of currents is needed, and therefore the voltage may be supplied from the capacitor 262 in the separating unit 26 if in a short time. As the separating unit 26 forms an RC circuit (low-pass filter circuit) by using the capacitor 262 and the resistor 261, a sufficiently reduced pulse signal is transmitted to the unit pixel 230. Furthermore, the pulse-signal detector 27 detects a pulse signal superimposed on the negative voltage due to AC coupling and outputs it to the timing generator 25.

Operation of the endoscope

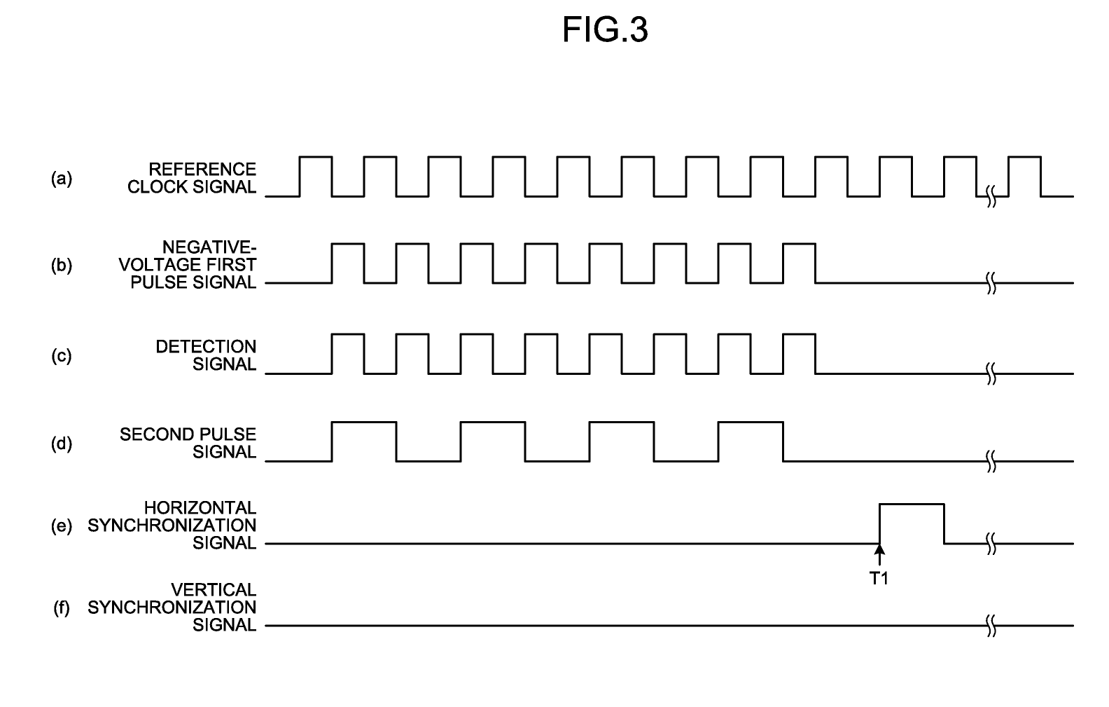

Next, the timing of each signal in the endoscope 2 is explained. FIG. 3 is a timing chart that illustrates the timing of each signal in the endoscope 2. In FIG. 3, sequentially from the top, (a) denotes the timing of the reference clock signal generated by the pulse-signal generator 54, (b) denotes the timing of the negative-voltage first pulse signal superimposed on the negative voltage by the pulse-signal superimposing unit 56, (c) denotes the timing of a detection signal (first pulse signal) detected by the pulse-signal detector 27, (d) denotes the timing of the second pulse signal obtained after conversion by the pulse-signal converting unit 28, (e) denotes the timing of a horizontal synchronization signal detected by the timing generator 25, and (f) denotes the timing of a vertical synchronization signal detected by the timing generator 25.

As illustrated in FIG. 3, the pulse-signal generator 54 doubles the frequency during the time period in which the negative-voltage pulse signal rises (the time period of High) and the number of times the negative-voltage first pulse signal rises (the number of times of High) and outputs it to the pulse-signal superimposing unit 56. Specifically, the pulse-signal generator 54 multiplies the frequency of the first pulse signal by an integer larger than one, i.e., by two, with respect to the frequency of the synchronization signal of the imaging unit 20 and outputs it to the pulse-signal superimposing unit 56. Therefore, the pulse-signal converting unit 28 divides the frequency of the first pulse signal by an integer larger than one, i.e., decreases it by ½, to convert it into the second pulse signal. This allows the timing generator 25 to generate a horizontal synchronization signal at timing T1.

Relationship Between the Filter Property of the Pulse-Signal Detector and a Frequency Component

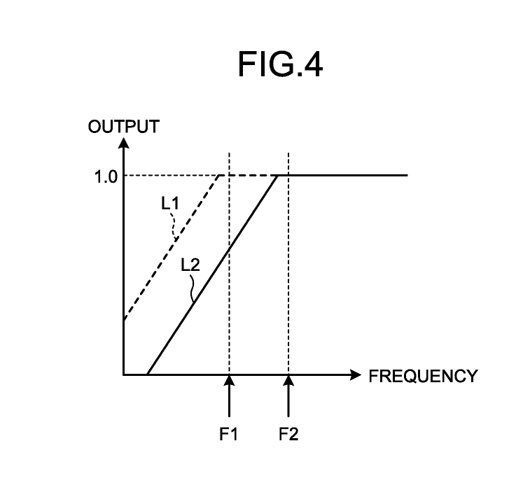

Next, an explanation is given of the relationship between the filter property of the pulse-signal detector 27 and the frequency component of the first pulse signal. FIG. 4 is a diagram that illustrates the relationship between the filter property of the pulse-signal detector 27 and the frequency component of the first pulse signal. In FIG. 4, the vertical axis indicates output (attenuation) of the pulse-signal detector 27, and the horizontal axis indicates a frequency. Furthermore, in FIG. 4, a polygonal line L1 indicates the relationship between the filter property of only the pulse-signal detector 27 and the frequency component, and a polygonal line L2 indicates the relationship between the frequency component and the filter property of the pulse-signal detector 27 when the pulse-signal converting unit 28 is provided at a subsequent step.

As illustrated in FIG. 4, the relationship between the filter property of the pulse-signal detector 27 and the frequency component of the first pulse signal enables a high cutoff frequency in the filter property of the pulse-signal detector 27 when the pulse-signal converting unit 28 is provided at a subsequent step, as indicated by the polygonal line L2, as compared with the filter property of only the pulse-signal detector 27 as indicated by the polygonal line L1. Specifically, the output that is the attenuation rate is 1.0 at a frequency F1 in the filter property of only the pulse-signal detector 27 as indicated by the polygonal line L1, while the output that is the attenuation rate is 1.0 at a frequency F2 (F1<F2) in the filter property of the pulse-signal detector 27 when the pulse-signal converting unit 28 is provided at a subsequent step, as indicated by the polygonal line L2. Thus, as the cutoff frequency (fc=½πRC) of the pulse-signal detector 27 may be set at a higher frequency side, the resistance values of the resistors 261, 272 included in the separating unit 26 and the pulse-signal detector 27, respectively, and the capacitances of the capacitors 262, 271 may be small. As a result, in the case of integration in the first chip 21 or the second chip 22, an increase in the area of the imaging unit 20 may be prevented.

According to the first embodiment of the disclosure described above, as the cutoff frequency of the pulse-signal detector 27 may be set at a higher frequency side, the resistance values of the resistors 261, 272 included in the separating unit 26 and the pulse-signal detector 27, respectively, and the capacitances of the capacitors 262, 271 may be small. As a result, in the case of integration in the first chip 21 or the second chip 22, an increase in the area of the imaging unit 20 may be prevented.

Furthermore, according to the first embodiment of the disclosure, the pulse-signal superimposing unit 56 superimposes the pulse signal for generating a synchronization signal to drive the imaging unit 20 on the transmission cable 3, through which the negative voltage is transmitted, and outputs it to the imaging unit 20, whereby the number of the transmission cables 3 connecting the imaging unit 20 and the connector portion 5 may be reduced.

Furthermore, according to the first embodiment of the disclosure, the timing generator 25 provided in the imaging unit 20 at the side of the distal end 101 generates a horizontal synchronization signal and a vertical synchronization signal based on the second pulse signal obtained after conversion by the pulse-signal converting unit 28 and the reference clock signal and transmits them to the first chip 21, whereby the signal line for transmitting synchronization signals may be reduced.

Moreover, according to the first embodiment of the disclosure, the separating unit 26, the pulse-signal detector 27, and the pulse-signal converting unit 28 may be integrated into the second chip 22. This allows a further reduction in the size of the imaging unit 20.

### Second Embodiment

Next, a second embodiment of the disclosure is explained. An endoscope system according to the second embodiment has a different configuration from the endoscope system 1 according to the above-described first embodiment. The configuration of the endoscope system according to the second embodiment is explained below. Furthermore, the same component as that of the endoscope system 1 according to the above-described first embodiment is attached with the same reference numeral, and explanation is omitted.

Configuration of the Endoscope System

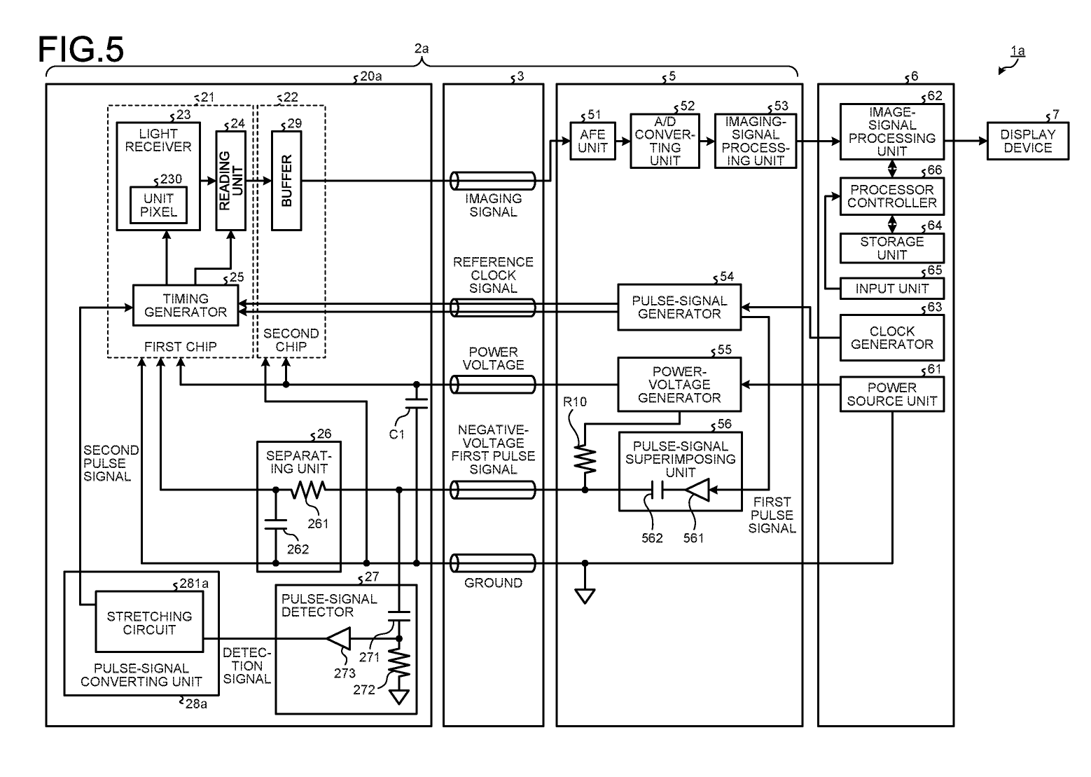

FIG. 5 is a block diagram that illustrates the function of a relevant part of the endoscope system according to the second embodiment of the disclosure. An endoscope system 1a illustrated in FIG. 5 includes an endoscope 2a instead of the endoscope 2 according to the above-described first embodiment. Furthermore, the endoscope 2a includes an imaging unit 20a instead of the imaging unit 20 according to the above-described first embodiment. Moreover, the imaging unit 20a includes a pulse-signal converting unit 28a instead of the pulse-signal converting unit 28.

The pulse-signal converting unit 28a converts the frequency of the first pulse signal detected by the pulse-signal detector 27 into the frequency of the second pulse signal used to generate a synchronization signal for driving the light receiver 23 and outputs it to the timing generator 25. The pulse-signal converting unit 28a includes a stretching circuit 281a that stretches the pulse width of the first pulse signal (detection signal) detected by the pulse-signal detector 27 by multiplying it by an integer larger than one to convert it into the second pulse signal. That is, the stretching circuit 281a stretches the pulse width of the first pulse signal by doubling it to convert it into the second pulse signal.

Operation of the Endoscope

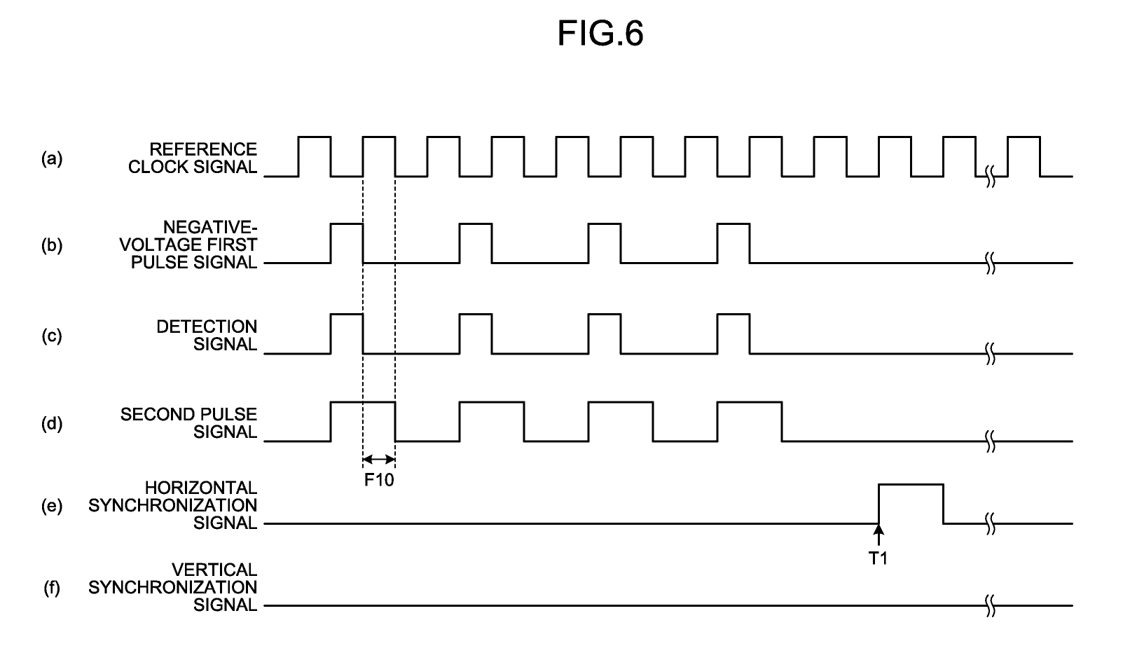

Next, the timing of each signal in the endoscope 2a is explained. FIG. 6 is a timing chart that illustrates the timing of each signal in the endoscope 2a. In FIG. 6, sequentially from the top, (a) denotes the timing of the reference clock signal generated by the pulse-signal generator 54, (b) denotes the timing of the negative-voltage first pulse signal superimposed on the negative voltage by the pulse-signal superimposing unit 56, (c) denotes the timing of the detection signal (first pulse signal) detected by the pulse-signal detector 27, (d) denotes the timing of the second pulse signal obtained after conversion by the pulse-signal converting unit 28a, (e) denotes the timing of the horizontal synchronization signal detected by the timing generator 25, and (f) denotes the timing of the vertical synchronization signal detected by the timing generator 25.

As illustrated in FIG. 6, the pulse-signal generator 54 multiplies the pulse width (the time period of High) of the negative-voltage first pulse signal during a rise by ½ and outputs it to the pulse-signal superimposing unit 56. That is, the pulse-signal generator 54 generates the first pulse signal by dividing the pulse width of the first pulse signal by an integer larger than one, i.e., decreasing it by ½, with respect to the frequency of the synchronization signal of the imaging unit 20 and outputs it to the pulse-signal superimposing unit 56. Therefore, the pulse-signal converting unit 28a stretches the pulse width (see F10 in FIG. 6) of the first pulse signal by multiplying it by an integer larger than one, i.e., doubling the pulse width, to convert it into the second pulse signal. This allows the timing generator 25 to generate a horizontal synchronization signal at the timing T1.

Thus, as the cutoff frequency (fc=½πRC) of the pulse-signal detector 27 may be set at a higher frequency side, the resistance values of the resistors 261, 272 included in the separating unit 26 and the pulse-signal detector 27, respectively, and the capacitances of the capacitors 262, 271 may be small. As a result, in the case of integration in the first chip 21 or the second chip 22, an increase in the area of the imaging unit 20a may be prevented.

According to the above-described second embodiment of the disclosure, as the same advantageous effect as that in the above-described first embodiment may be produced and the cutoff frequency of the pulse-signal detector 27 may be set at a higher frequency side, the resistance values of the resistors 261, 272 included in the separating unit 26 and the pulse-signal detector 27, respectively, and the capacitances of the capacitors 262, 271 may be small. As a result, in the case of integration in the first chip 21 or the second chip 22, an increase in the area of the imaging unit 20a may be prevented.

Furthermore, according to the second embodiment of the disclosure, the separating unit 26, the pulse-signal detector 27, and the pulse-signal converting unit 28a may be integrated into the second chip 22. This allows a further reduction in the size of the imaging unit 20a.

### Other Embodiments

Furthermore, although the processor and the light source device are separated according to the embodiment of the disclosure, this is not a limitation and, for example, the processor and the light source device may be integrally formed.

Furthermore, although each of the separating unit, the pulse-signal detector, and the pulse-signal converting unit is provided at the distal end side of the insertion portion according to the embodiment of the disclosure, each of the separating unit, the pulse-signal detector, and the pulse-signal converting unit may be laminated on the second chip. Specifically, the imaging element (imaging unit) includes: a first chip having a light receiver including a plurality of pixels laminated thereon; and a second chip laminated on the first chip, and the second chip may include: a separating unit that separates an offset voltage and a pulse voltage from the voltage on which the first pulse signal is superimposed via the transmission cable through which the electric power (power voltage) for driving the first chip is transmitted and that outputs the offset voltage to the first chip; a pulse-signal detector that detects the first pulse signal superimposed on the transmission cable; a pulse-signal converting unit that converts the frequency of the first pulse signal detected by the pulse-signal detector into the frequency of the second pulse signal used to generate a synchronization signal for driving the first chip; and a timing generator that generates a synchronization signal based on the second pulse signal obtained after conversion by the pulse-signal converting unit.

Furthermore, according to the embodiment of the disclosure, the pulse-signal superimposing unit may superimpose the pulse signal on the negative voltage at the negative side (−side) or the positive side (+side).

Furthermore, although the power voltage generator transmits the negative voltage via the transmission cable according to the embodiment of the disclosure, this is not a limitation, and the positive voltage may be transmitted.

Furthermore, although each of the separating unit, the pulse-signal detector, and the pulse-signal converting unit is provided at the distal end side of the insertion portion according to the embodiment of the disclosure, the separating unit, the pulse-signal detector, and the pulse-signal converting unit may be provided in the operating unit of the endoscope.

Furthermore, although an explanation is given in the embodiment of the disclosure by using the simultaneous lighting endoscope as an example, a sequential lighting endoscope is also applicable.

Furthermore, according to the embodiment of the disclosure, endoscope systems of rigid endoscopes, sinus endoscopes, electric cauteries, examination probes, or the like, are also applicable other than flexible endoscopes (upper and lower endoscopy scopes).

Furthermore, for explanations of the timing charts in this description, a sequential order of each process is indicated by using terms such as “first”, “next”, and “then”; however, the sequential order of a process necessary to implement the disclosure is not uniquely defined by using those terms. That is, the order of a process in the timing chart described in this description may be changed to such a degree that there is no contradiction.

Furthermore, in the description and drawings, if a term is described together with a different term having a broader meaning or the same meaning at least once, it may be replaced with the different term in any part of the description or drawings. Thus, various modifications and applications are possible without departing from the scope of the disclosure.

According to the disclosure, there is an advantage that it is possible to achieve a further size reduction.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging device comprising:
a light receiver that includes a plurality of pixels that is arranged in a two-dimensional matrix to receive light from outside and generate an imaging signal corresponding to an amount of received light;
a transmission cable through which electric power is transmitted to the light receiver;
a power source configured to supply a voltage to the light receiver via the transmission cable;
a pulse-signal generator configured to generate a first pulse signal;
a pulse-signal superimposing circuit configured to superimpose the first pulse signal generated by the pulse-signal generator on the voltage;
a separating circuit that is disposed between the light receiver and the transmission cable, the separating circuit being configured to separate an offset voltage and a pulse voltage from the voltage transmitted through the transmission cable and output the offset voltage to the light receiver;
a pulse-signal detector configured to detect the first pulse signal superimposed on the transmission cable;
a pulse-signal converting circuit configured to convert a frequency of the first pulse signal detected by the pulse-signal detector into a frequency of a second pulse signal used to generate a synchronization signal for driving the light receiver; and
a timing generator configured to generate the synchronization signal based on the second pulse signal obtained after conversion by the pulse-signal converting circuit.

2. The imaging device according to claim 1, wherein the pulse-signal converting circuit includes a frequency divider circuit configured to divide the frequency of the first pulse signal by an integer larger than one to convert the first pulse signal into the second pulse signal.

3. The imaging device according to claim 1, wherein the pulse-signal converting circuit includes a stretching circuit configured to stretch a pulse width of the first pulse signal by multiplying the pulse width by an integer larger than one to convert the first pulse signal into the second pulse signal.

4. The imaging device according to claim 1, wherein the power source and the pulse-signal superimposing circuit are located at a proximal end side of the transmission cable, and the separating circuit, the pulse-signal detector, the pulse-signal converting circuit, and the timing generator are located at a distal end side of the transmission cable.

5. An endoscope comprising:
the imaging device according to claim 1;
an insertion portion that is insertable into a body of a subject; and
a connector portion that is attachable to and detachable from an image processing device configured to perform image processing on the imaging signal, wherein
the light receiver, the separating circuit, the pulse-signal detector, the pulse-signal converting circuit, and the timing generator are located at a distal end side of the insertion portion, and
the connector portion includes the power source and the pulse-signal superimposing circuit.

6. An endoscope system comprising:
the endoscope according to claim 5; and
an image processing device configured to perform image processing on the imaging signal.
